(19) United States
(12) Patent Application Publication
(10) Pub. No.: US 2005/0113701 A1
(43) Pub. Date: May 26, 2005

(54) Rotating measuring device

(75) Inventors: Yem Chin, Burlington, MA (US); Louis J. Barbato, Franklin, MA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: SCIMED Life Systems, Inc.

(21) Appl. No.: 10/723,847

(22) Filed: Nov. 26, 2003

(51) Int. Cl. A61B 6/00

(52) U.S. Cl. 600/478

(57) ABSTRACT

A system for measuring internal body cavities of a patient includes an interferometer having a reference leg and a patient leg that is inserted in the patient. The patient leg directs a beam of coherent light within a body cavity of the patient. Light exiting the patient leg is reflected by a tissue wall and is received by the patient leg where it is combined with light that is reflected through the reference leg. The light in the reference leg and patient leg combines to create fringes indicative of a difference in the optical path length between the two legs. A processor computes the dimensions of the body cavity from the difference in optical path length between the patient leg and the reference leg.

### FIELD OF THE INVENTION

The present invention relates to medical devices, and in particular, to devices for measuring internal body cavities.

### BACKGROUND OF THE INVENTION

In many medical procedures, it is desirable to know the dimensions of a particular portion of a patient's anatomy. Such information may be used to properly select a medical device that will be placed in the body. Alternatively, a physician may be tracking a disease or other physiological process where it is useful to take bodily measurements.

Numerous techniques are known for measuring anatomical features. For example, it is known to use an ultrasound transducer to measure the size of blood vessels, cardiac chambers, fetal growth, etc. However, the use of ultrasound is limited to those locations where a fluid is present between the ultrasound transducer and the target anatomy to be measured. Other external imaging techniques such as x-rays or magnetic resonance imaging (MRI) can be used to measure portions of a patient's body. However, these tools are relatively expensive and use machines that are not very portable.

Given these problems, there is a need for a simple mechanism of measuring anatomical features that is both accurate and relatively inexpensive. Furthermore, the system should be relatively small and portable.

### SUMMARY OF THE INVENTION

To address the above-mentioned concerns, the present invention is an imaging system that measures anatomical features of a patient. The system includes a coherent light source and a beam splitter that divides light from the light source into a reference leg and a movable patient leg that is inserted into a patient. An interferometer includes a detector that detects constructive and destructive fringes in light that is combined from the reference and patient legs. The fringes are counted to determine an optical path length difference between light that is transmitted in the patient leg and the light that is transmitted in the reference leg.

An imaging system of the present invention includes a mechanism for rotating the patient leg or the light emitted from the patient leg within the patient. Light exits the patient leg and is reflected off a wall of the patient's anatomy. The reflected light returns through the patient leg where it is combined with light reflected through the reference leg to determine the difference in the optical path length between the reference and the patient legs.

The patient leg may be marked with a visual or other detectable indication of distance along its length so that the depth of insertion of the patient leg into the patient can be determined. The depth information and measurement information provided by the interferometer can be used to construct a three-dimensional image or model of the patient's anatomy.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 illustrates a system for measuring portions of a patient's anatomy in accordance with one embodiment of the present invention;

FIG. 2 is a block diagram of a rotating interferometer used in the measuring system of the present invention; and

FIG. 3 illustrates one embodiment of a quadrature detector used with the present invention.

### DETAILED DESCRIPTION OF THE PREFERRED EMBODIMENT

As indicated above, the present invention is a system for measuring the anatomy of a patient and in particular for measuring internal body cavities of a patient. Such cavities may include a patient's esophagus, uterus, colon, nasal cavities, or other areas having an air gap that extends between a patient leg and walls of the cavity.

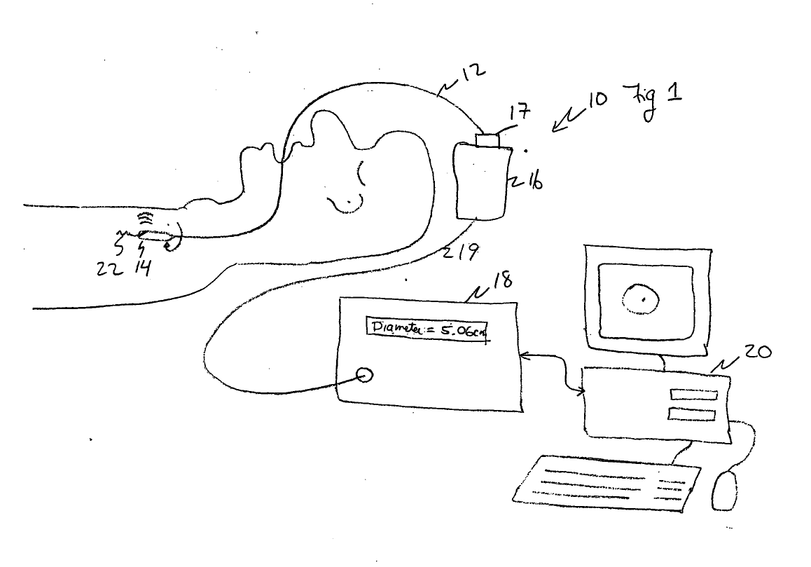

FIG. 1 illustrates one embodiment of a measuring system in accordance with the present invention. The system 10 includes patient leg 12 that is insertable into a patient. The patient leg 12 directs a rotating beam of coherent light within the patient's body cavity. The coherent light beam exits the patient leg at an angle such as 90 degrees with respect to the longitudinal axis of the patient leg 12. The light emitted from the patient leg reflects off a tissue wall and is picked up by the patient leg where it is transmitted in the opposite direction through the patient leg. The patient leg includes one or more glass or plastic, single or multi-mode, optical fibers to carry the light. The tips of the optical fibers are either polished to emit and receive light at the desired angle or are coupled to one or more lenses to emit and receive the light.

The optical fibers of the patient leg 12 are included within a catheter or endoscope that can be inserted directly into the patient's body. Alternatively, the catheter can include a guidewire lumen for routing the catheter over a guidewire 22. The system 10 further has a mechanism 16 that includes a rotating optical coupler 21 to allow the optical fibers of the patient leg 12 to rotate within the patient's body such that the light emitted sweeps around the body cavity. The mechanism 16 may include a motor 17 or may be hand-turned to rotate the optical fibers within the catheter or the catheter and fibers together. The optical fibers of the patient leg 12 are coupled to a rotatable lens that may include a ball lens or GRIN lens such that light can be transmitted into and received from the optical fibers as they are rotated. In addition, the mechanism 16 may provide an indication of the angular position of the one or more optical fibers. As an alternative, the mechanism 16 rotates the catheter through which the optical fibers are routed and the optical fibers together or the mechanism 16 may move a mirror or other light directing mechanism at the distal end of the patient leg 12 to direct the light within the body cavity.

The system 10 also includes a control box 18 that delivers light to the patient leg 12 and receives light that is reflected off the cavity wall. The control box 18 preferably includes a processor and a display for calculating and displaying the dimensions of a body cavity as will be described below. In some embodiments, the mechanism 16 for rotating the patient leg may be found within the control box 18. The control box 18 may be connected to a computer system 20 that receives the information regarding the dimensions of the body cavity or that receives the data used to compute the dimensions in order to produce a two-dimensional representation of the body cavity that is shown on a video monitor.

In addition, the computer system 20 may receive information regarding the depth at which the patient leg 12 has been inserted into the patient in order to construct a 3D model or map of the patient's body cavity. The depth information may be visually determined based on length marks imprinted along the patient leg 12. In this case, an operator reads the depth and enters the data into the computer 20 where it is combined with the dimension information in order to produce a three-dimensional map or model of the body cavity. Alternatively, the patient leg 12 may include machine-readable markings that are sensed by a sensor (not shown) and fed to the computer system 20 in order to determine the depth of insertion information automatically.

Based on the dimensions of the body cavity, the physician can gain insight into the internal structure of the body and can, for example, select an appropriately sized device for implantation into the patient. One example of a medical device that must be correctly sized is an esophageal stent that is placed in the esophagus to keep a passageway to the stomach open. By knowing the dimensions of the patient's esophagus, the physician can select the correctly sized stent without trial and error.

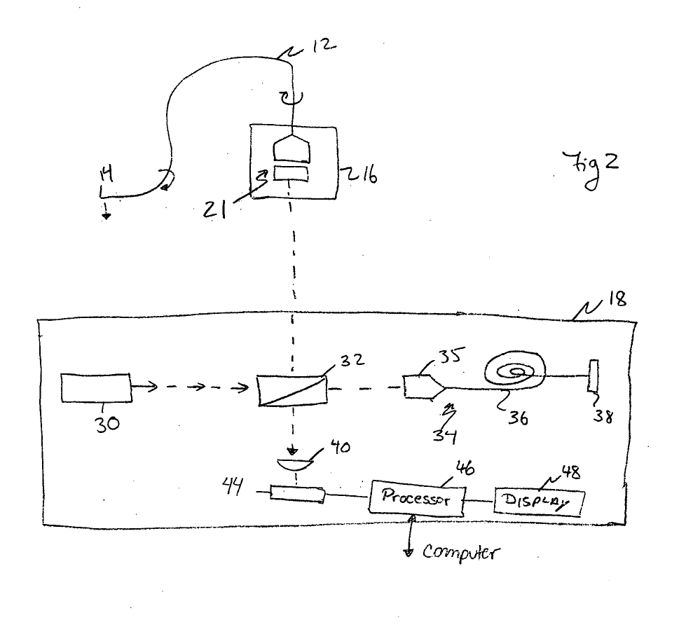

FIG. 2 shows additional detail of one embodiment of the measuring system of the present invention. Within the control box 18 is a light source 30 that preferably produces a highly coherent light such as laser light. Light from the light source is directed to a fiber optic beam splitter 32 that directs a portion of the light beam into a reference leg 34 and a portion into the patient leg 12.

In the reference leg 34, light is directed through a fiber optic coupler 35 to a known length of one or more optical fibers 36 that are terminated with a mirror 38. Light is reflected off the mirror 38 and returns through the one or more optical fibers 36 back to the fiber optic beam splitter 32. Light that is returned through the reference leg 34 is directed by the fiber optic beam splitter 32 to a lens 40 that focuses the light on a detector 44.

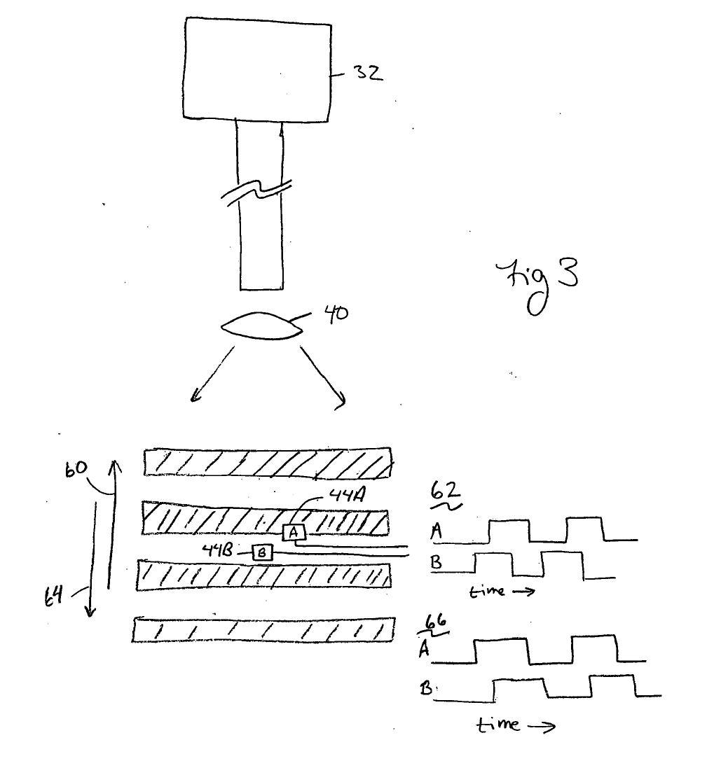

In a similar fashion, some of the light from the light source 30 is directed by the beam splitter 32 through a rotating optical coupler 21 and into the one or more optical fibers of the patient leg 12. As indicated above, the light produced at the distal end of the patient leg is rotated such that the light travels around the circumference of the body cavity in which the patient leg is inserted. The light in the patient leg 12 is reflected off the cavity wall and returns through the one or more optical fibers of the patient leg 12 to the fiber optic beam splitter 32. The light passes through the fiber optic beam splitter 32 where it is directed to the lens 40 that focuses the light onto the detector 44. As shown in FIG. 3, the fiber optic beam splitter 32 directs the combined light from the patient and reference legs towards the lens 40. The lens 40 spreads out the pattern of light and dark fringes over a pair of light detectors 44A and 44B. The detectors are preferably spaced in quadrature given the wavelength of light produced by the coherent light source. The detectors 44A and 44B produce signals that indicate the number and direction of movement of the fringes. If the fringes move in a direction as indicated by the arrow 60, then the detectors will produce signals like those illustrated at 62. Alternatively, if the fringes move in a direction as indicated by the arrow 64, then the detectors will produce signals like those illustrated at 66. The optical path length of the reference leg 34 and the patient leg 12 are preferably equivalent such that the fringes that are detected by the detectors 44A, 44B are dependent on the distance between the point at which the light exits the patient leg and the tissue wall that reflects the light back to the patient leg.

As will be appreciated, the detectors 44A, 44B produce a series of pulses that depend on the distance between the patient leg and the tissue wall that reflects light back to the patient leg. If the body cavity is cylindrical and the patient leg is positioned at the center of the cylinder of the cavity, the counts are directly proportional to the radius of the body cavity. The diameter of the body cavity can therefore be determined by doubling the radius detected.

In most cases, the cross-sectional profile of the body cavity is not perfectly round, and it cannot be guaranteed that the patient leg is always positioned midway between opposite sides of the cavity walls. In this case, the diameter of the cavity can be determined by adding the radius measurements taken at positions that are 180 degrees apart in the body cavity. The light that exits the patient leg should be rotated in the body cavity at a sufficient rate such that the position of the patient leg does not move significantly between the time when the light is directed to opposite walls of the body cavity.

Based upon the detector counts and knowing the angular position of the rotating light beam, the processor 46 calculates the dimensions of the internal body cavity to a high degree of accuracy. The processor 46 may display the dimensions on a dedicated display 48 on the control box 18. Alternatively, the processor 46 may interface with the computer 20 to display the dimensions and/or construct a three-dimensional model of the body cavity.

As can be seen from the above description, the present invention is a simple and highly accurate mechanism for detecting dimensions of internal body cavities that are not filled with a fluid. The system is inexpensive enough to allow the patient leg and/or the reference leg to be disposable and is portable enough to be used in a variety of settings within a clinic or hospital. Furthermore, the system does not subject the patient to x-rays or other potentially high-energy radiation sources.

While the preferred embodiment of the invention has been illustrated and described, it will be appreciated that various changes can be made therein without departing from the scope of the invention. It is therefore intended that the scope of the invention be determined from the following claims and equivalents thereof.

1. A system for measuring dimensions of a body cavity, comprising:
a source of coherent light;
a patient leg that is insertable into a patient's body cavity including one or more optical fibers that direct light from the light source against a wall of the body cavity and receive light reflected from the cavity wall;
a reference leg including one or more optical fibers and a reflecting surface at an end thereof that receives light from the light source and reflects the light at the end of the reference leg;
a beam splitter that divides light from the light source into the patient leg and the reference leg and combines light reflected from the reference leg and the patient leg;
at least two detectors spaced in quadrature for detecting fringes in the combined light; and
a processor for counting the fringes detected by the detector, wherein the number of fringes is proportional to the distance between an end of the one or more optical fibers in the patient leg and the wall of the body cavity.

2. The system of claim 1, further comprising a mechanism for rotating the light emitted by the one or more optical fibers in the patient leg within the body cavity.

3. The system of claim 2, wherein the mechanism for rotating the light includes a rotatable optical coupler that couples light into the one or more optical fibers of the patient leg and a motor that rotates the one or more optical fibers in the patient leg.

4. The system of claim 3, wherein the one or more optical fibers in the patient leg are routed in a catheter and the mechanism for rotating the light rotates the catheter.

5. The system of claim 2, wherein the mechanism for rotating the light within the body cavity includes a movable light deflector at or adjacent the distal end of the patient leg that directs the light emitted within the body cavity.

6. The system of claim 1, wherein the one or more optical fibers of the patient leg are routed within a catheter that includes indications of length thereon, the system further including a computer that receives information regarding a depth of insertion of the catheter to construct a model of the body cavity.

7. The system of claim 6, where the indications of length on the catheter are visually perceptible.

8. The system of claim 6, wherein the indications of length on the catheter are machine-readable.

9. A system for measuring an internal body cavity of a patient, comprising:
an interferometer that directs a beam of coherent light into a reference leg and a patient leg that is insertable into the body cavity, the patient leg including a length marking that indicates the depth of insertion of the patient leg into the body cavity;
a mechanism for rotating the beam of light within the body cavity and receiving light that is reflected from a wall of the body cavity;
a detector that detects a difference in an optical path length between light directed into the reference leg and the patient leg; and
a computer system that receives signals from the detector and an indication of the depth of insertion of the patient leg to construct a three-dimensional model of the body cavity.

10. The system of claim 9, wherein the length marking on the patient leg is readable by a sensor connected to the computer.

11. The system of claim 9, wherein the detector includes at least a first and second sensor positioned in quadrature.
